(19) United States
(12) Patent Application Publication
(10) Pub. No.: US 2010/0080849 A1
(43) Pub. Date: Apr. 1, 2010

(54) PELLETS CONTAINING A PHARMACEUTICAL SUBSTANCE, METHOD FOR THE PRODUCTION THEREOF AND USE OF THE SAME

(75) Inventor: Burkhard Schlutermann, Au (DE)

Correspondence Address: HEDMAN & COSTIGAN P.C., 1185 AVENUE OF THE AMERICAS, NEW YORK, NY, 10036, US

(73) Assignee: Add Advanced Drug Delivery Technologies Ltd., Reinach (CH)

(21) Appl. No.: 12/449,946

(22) PCT Filed: Mar. 13, 2008
(86) PCT No.: PCT/EP2008/002026

§ 371 Date: Nov. 30, 2009

(30) Foreign Application Priority Data
Mar. 13, 2007 (DE) 10 2007 012 105.0

(51) Int. Cl. A61K 9/16; A61K 31/381; A61P 25/24

(52) U.S. Cl. 424/489; 514/438

(57) ABSTRACT

The invention relates to pellets containing a pharmaceutical substance with a breaking strength of more than 0.001 newton, method of production thereof and pharmaceutical preparations based on said pellets.

The invention relates to pellets containing a pharmaceutical substance with a breaking strength of more than 0.001 newton, method of production thereof and pharmaceutical preparations based on said pellets.

Pellets containing pharmaceutical substances are known. For example there are pharmaceutical pellets that contain a so-called starter core based on an excipient and coated with a layer that contains a pharmaceutical active substance. Sucrose crystals or spherical bodies of an excipient combination of sucrose and starch, for example, have been proposed as starter cores for these purposes. In addition, there are particles based on microcrystalline cellulose. Moreover, it is known that spherical, especially rapidly disintegrating bodies can be produced by special fluidized-bed aggregation methods, and can contain water-soluble carbohydrates, among others, as main constituent.

The properties of the starter cores already known are not, however, always satisfactory. Furthermore, the established production methods for starter pellets based on sucrose are very time-consuming and cost-intensive. In particular the known starter cores have the drawback that they have unfavorable dietetic and cariogenic properties, do not always display sufficient mechanical stability, and can absorb water at low air humidity.

In the case of chemically labile or unstable active substances, a low water content and/or a lower water absorbency of said pellets can lead to improved processing and stability behavior.

Generally, excipients that are well-tolerated and are acceptable to diabetic patients should also be used increasingly.

There is therefore a need to develop new production processes and starter pellets with an improved product profile. There is also a need to develop pellets that contain pharmaceutical active substances.

With the present invention, pellets are now to be provided that can be used advantageously for the production of pharmaceutical preparations.

In particular, pellets are to be provided that have high mechanical strength. Furthermore, pellets are to be provided that are water-soluble and/or have low hygroscopicity.

Another problem to be solved by the invention is to provide a method of production of said pellets.

In particular, a method is to be provided that makes it possible to keep the pellets according to the invention as spherical as possible over a wide particle-size range. The pellets obtained should moreover be suitable in particular for further processing to oral dosage forms, in particular with a modified release profile.

The resultant products should allow distribution of a pharmaceutical active substance to many subunits, which from the bio-pharmaceutical standpoint is advantageous in particular for modified-release preparations, since products based on this formulation concept show little variability of the pharmacokinetic parameters.

The present invention should also provide an oral pharmaceutical preparation that offers improved preconditions for controlled, robust further processing, in particular the application of coatings.

According to the invention, it was found that suitable pellets can be built up from individual droplets of a dispersion, if the droplets are carried by means of a suitably preheated process gas stream so that particles from an already solidified dispersion can come in contact again with droplets. In contrast to the production of particles from a dispersion in a conventional spraying tower, the particles that form are circulated in a suitable processing space until as a result of repeated deposition of droplets of the dispersion they have reached the desired size.

The invention therefore relates to a method of production of pellets containing a pharmaceutical substance, preferably with a breaking strength of more than 0.001 newton and in particular more than 0.05 newton (abbreviated to “N” hereinafter), comprising the stages:
(a) preparation of a solution or dispersion of a pharmaceutical substance;
(b) production of droplets by spraying the solution or dispersion into a processing space;
(c) repeated directing of solid particles past injected droplets in the processing space by means of a process gas stream conveyed in a preferentially defined manner;
(d) removing particles from the processing space in relation to particle size.

The invention also relates to pellets that contain a pharmaceutical substance and preferably have a breaking strength of more than 0.001 newton and in particular more than 0.05 newton.

The expression “pharmaceutical substance” denotes pharmaceuticals, excipients and mixtures of said components.

According to a preferred embodiment the pharmaceutical substance contains an excipient or mixtures of excipients, in particular it consists thereof. Excipients that are described in Fiedler: “Encyclopedia of excipients”, 5th edition 2002, Editio Cantor Verlag, are preferably used. Within the scope of this application, urea is not regarded as a pharmaceutical substance.

Examples of excipients are for example binders, fillers and/or disintegrants.

Preferably it is a water-soluble and/or nonhygroscopic excipient. Furthermore, preferred excipients are sucrose-free.

Water-soluble is to be understood as water solubility of more than 50 mg per liter at 20° C., preferably more than 200 mg per liter at 20° C.

A substance is classified as “nonhygroscopic” if on storage for 24 hours at a relative humidity of 50% and a temperature of 20° C. it absorbs not more than 10 wt. %, in particular not more than 5 wt. % water.

Examples of preferred excipients are mono-, di- or polysaccharides and/or sugar alcohols, for example arabinose, ribose, xylose, glucose, mannose, galactose, fructose, lactose, maltose, trehalose, isomalt, inulin, mannitol, sorbitol, xylitol, maltitol, erythritol and mixtures thereof.

In particular mannitol is used as excipient.

Preferably the pellets according to the invention contain the aforementioned excipients, in particular mannitol, in an amount of more than 50 to 100%, in particular from 80 to 100%. According to a preferred embodiment the pellets consist substantially of the excipient, especially of mannitol.

In an alternative embodiment the pharmaceutical substance contains a pharmaceutical active substance. Basically this can be any pharmaceutical that is solid at room temperature. Optionally the pharmaceutical is not enzymes or ibuprofen.

The pellets according to the invention, which contain an excipient, are in a preferred embodiment so-called starter pellets, also known as “starter spheres” in English. The starter pellets are also called cores. According to a preferred embodiment the cores consist essentially of the excipient, especially of mannitol. Cores that contain mannitol or preferably consist essentially of the excipient mannitol, are called mannitol cores. The starter pellets are usually coated with a layer containing a medicinal product. In particular, starter pellets containing mannitol are preferred.

The pellets according to the invention are preferably spherical. According to the invention, a particle is designated as spherical if the length-width ratio (i.e. the ratio of the length (largest dimension) of the particle, divided by the width (smallest dimension, determined at an angle of 90° to the length) is less than about 1.4. Preferably the length-width ratio of a spherical particle is less than about 1.3, more preferably less than about 1.2, even more preferably less than about 1.1 and in particular less than about 1.05. The particles are in addition characterized by their size.

The particle size distribution can be determined by sieve analysis. Unless stated otherwise, the particle size refers to the weight average.

In a preferred embodiment the weight average particle diameter (D₅₀) is 0.1 to 3 mm, more preferably from 0.15 to 1 mm. Depending on intended use, alternatively weight average particle diameters of for example 150 to 350 μm or 300 to 500 μm may be preferred.

Furthermore, the pellets according to the invention preferably have a narrow grain size distribution. The grain size distribution is characterized by the ratio D₁₀/D₉₀. Within the scope of the invention, the ratio is preferably 0.4 to 1, more preferably 0.5 to 1, in particular 0.6 to 1.

Here, D₁₀ is the particle size at which 10% of the particles are smaller than this particle size, D₅₀ is the particle size at which 50% of the particles are smaller than this particle size, and D₉₀ is the particle size at which 90% of the particles are smaller than this particle size.

The pellets according to the invention can in addition have a preferred bulk density from 0.5 to 1 g/ml, more preferably from 0.6 to 0.8 g/ml.

The pellets according to the invention preferably have a breaking strength of more than 0.001 N. More preferably the pellets according to the invention have a breaking strength of more than 0.005 N, 0.01 N, 0.05 N, 0.1 N, 0.15 N, 0.2 N, 0.25 N, 0.3 N, 0.35 N, 0.4 N, 0.45 N, 0.5 N, 0.55 N, 0.6 N, 0.65 N, 0.7 N, 0.75 N, 0.8 N, 0.85 N, 0.9 N, 0.95 N, 1 N, 1.5 N, 2 N, 2.5 N, 3 N, 3.5 N, 4 N, 4.5 N or alternatively 5 N. Usually the breaking strength is not more than 25 N or alternatively 10 N. In a preferred embodiment the pellets have a breaking strength from 0.05 N to 10 N.

The breaking strength is determined as follows:

The breaking strength of the spherical specimens is measured on individual specimens by uniaxial vertical loading between two horizontal jaws with plane-parallel surfaces. The two jaws consist of pieces of a commercial (100) silicon wafer. The breaking strength is measured by recording a controlled force-distance curve with a resolution of 2 mN in the range from 0 to 25 N.

The specimen is placed on the lower jaw, and then the upper jaw is lowered progressively. The force-distance curve shows a first bend on first contact of the specimen with the upper jaw and reflects in the first section the orientation of the possibly not perfectly spherical specimen between the two jaws. Starting from this point, for the time being the elastic response of the specimen is measured. When the breaking strength is exceeded, there is a sudden decrease in the force applied at the controlled distance. The force present immediately before breakage is called the breaking strength here (in the present case in units of N, newton).

The breaking strength may depend on the average particle diameter. In a preferred embodiment the quotient of breaking strength to weight average particle diameter is 0.005 to 10 [N/mm], more preferably 0.05 to 5 [N/mm], in particular 0.5 to 3 [N/mm].

The invention also covers any combination of the embodiments described above.

The present invention also relates to a product that comprises a plurality of particles. Said product comprises a collection of particles, typically 50 or more, preferably 100 or more particles. A product according to the invention mainly comprises particles that fulfill the particle criteria according to the invention. Preferably at least 90%, in particular at least 95% and quite especially preferably at least 98% of the particles have the properties described above with respect to breaking strength, weight average particle diameter and length-width ratio.

The pellets described above can be produced by the method according to the invention. As already explained, it was found according to the invention that it is possible to produce spherical particles from individual droplets of a dispersion.

The particles preferably have a compact structure. Furthermore, it is preferable if the particles have a homogeneous surface structure (surface texture). It is important that the formation of spherical particles is made possible, with supplied particles or particles formed from the dispersion coming into contact repeatedly with droplets of the dispersion, so that spherical particles of a desired size can be built up. For this, the particles are moved within a processing space by means of a process gas stream conveyed in a defined manner. Particles that have reached a desired size can leave the processing space. The process gas stream is important both for mass transport and for heat transport.

The method according to the invention comprises stages (a) to (d).

In stage (a) of the method according to the invention, a solution or dispersion of a pharmaceutical substance is prepared.

Regarding the expression “pharmaceutical substance”, reference is to be made to the details given above concerning the pellets according to the invention.

The pharmaceutical substance is prepared as a solution or dispersion by dissolving or dispersing in a solvent. The above explanations for the pharmaceutical substance of the pellets according to the invention apply to the pharmaceutical substance used in the method according to the invention. Accordingly, in a preferred embodiment excipients are dissolved or dispersed, more preferably the previously stated saccharides and/or sugar alcohols; in particular mannitol.

The expression “dispersion” is to be understood in its broad sense within the scope of this invention. It comprises solid/liquid and liquid/phase systems, i.e. the term dispersion comprises suspensions and emulsions.

Water is preferably used as dispersant for solutions, emulsions or suspensions. Organic solvents or mixtures of two or more organic solvents are also possible as dispersants. Examples of suitable organic solvents are alcohols, esters or chlorinated solvents such as methylene chloride. Moreover, mixtures of water with organic solvents are possible. Examples are water/alcohol or water/ethyl acetate. Preferably water is used as the only solvent.

In stage (b) of the method according to the invention, droplets are produced by spraying (injection) of the solution or dispersion into a processing space.

Injection of the solution or dispersion usually takes place at elevated temperature. The temperature depends on the nature of the substance to be processed. The injection temperature should be at least 40° C. below the melting point of the pharmaceutical substance. In a preferred embodiment the solution or dispersion is injected at a temperature from 10 to 150° C., especially from 30 to 120° C., and in particular from 60 to 100° C.

In a preferred embodiment an injection rate from 20 to 200, in particular from 30 to 50 g/min is used.

In stage (c) of the method according to the invention, solid particles are again directed past injected droplets in the processing space by means of a process gas stream conveyed in a preferentially defined manner.

According to the invention a process gas stream is used for passing solid particles repeatedly past injected droplets. The process gas can for example be air or an unreactive gas, such as nitrogen, carbon dioxide or an inert gas.

In particular the temperature conditions provided in the processing space are such that solidification of the droplets is sufficiently delayed to allow wetting of the already solid particles with the injected droplets of the dispersion and the formation of spherical structures. Moreover, according to the invention, contact between particles with a liquid surface, resulting in sticking together, is largely avoided.

Accordingly, the process gas stream has a temperature between 10° C. and 150° C., preferably from 60° C. to 110° C.

According to the invention it is preferable to bring droplets of the dispersion and solid particles in contact with one another in a spouted bed. “Spouted bed” means that the completely fluidized solid particles are in a time-stable closed solid stream. The spouted bed is produced by means of the process gas stream that is conveyed in a defined manner. Three fluidization states or zones can be distinguished in the spouted bed. In a first zone or ejection zone the solid particles are accelerated under the action of the process gas stream that is conveyed in a defined manner, with the particles moving in this zone in the direction of flow of the process gas stream. Typically the process gas stream is directed vertically upward. Accordingly, a flow that is directed vertically upward prevails in the ejection zone of the spouted bed. In a subsequent second zone or fountain zone the particles change their direction of flow. A cross flow prevails. Finally the particles enter a third zone or return zone. There, the particles then move in the opposite direction, until they finally again come under the influence of the gas stream that is conveyed in a defined manner and are entrained by this back into the first zone. In the return zone the particles typically move under the effect of gravity. The dispersion can be atomized by two-component and multicomponent nozzles. Furthermore, it is possible for atomization to be brought about by pressure nozzles. Alternatively, droplets can be produced by means of rotary atomizers, stream cutters, ultrasonic splitters and other devices known by a person skilled in the art.

According to the invention, through injection of droplets of a solution or dispersion into the processing space and solidification of said droplets, it is possible for “seeds” to form from solid particles, which are then brought in contact with other droplets to form particles of the desired size. Alternatively or additionally, in this process solid particles can be supplied from outside. For example, particles that are too small and are removed from the process can be returned to the processing space as seed material. Similarly, particles or agglomerates of particles that are too large and are removed from the process can be comminuted by any comminution device and returned to the processing space as seed material.

In stage (d) of the method, the particles formed are removed from the processing space, depending on the particle size. Discharge of finished product from the processing space or material transport to another subsequent processing space can take place in the region of the transition of the cross flow to the downward-directed solid stream. According to one embodiment, the particles removed from the processing space are not classified.

According to another embodiment, the particles removed from the processing space are classified by means of one or more screening devices.

The method according to the invention can for example be carried out by means of a device as described in DE 103 22 062 A1 (zigzag classifier). The contents of this application are included in the present application by reference.

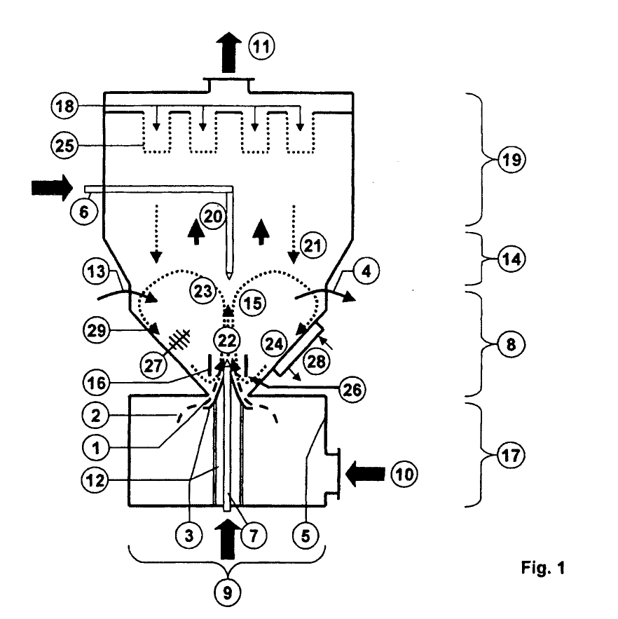

Preferably the method according to the invention is carried out using a device as shown in the appended FIG. 1. This is explained in detail below.

The amount of process gas 10 (as a rule heated air) required for solidification of the particles to be produced is supplied to an ingoing air chamber 17 with rectangular cross-section 9 and delimiting side walls 5. In the ingoing air chamber 17 the process gas 10 is distributed and travels via slits 1 into the processing space 8 in the form of gas streams 2. The process gas flow entering slit 1 preferably horizontally is deflected by deflector 3 preferably upward into the processing space 8 and flows as a kind of free jet into the apparatus. Furthermore, the apparatus cross-section can optionally increase in size in the expansion zone 14, so that the velocity of the process gas flow gradually decreases in the upward direction. The gas leaves the apparatus as waste gas 11 above the expansion zone 14 via the air outlet 19, in which optionally a dust-collecting system (e.g. filter cartridges or textile filter elements) can be integrated.

In the processing space 8 there is an amount of particles, which are entrained in the upward direction by the process gas stream. In the upper region of the processing space 8 and in the expansion zone 14 located above that, the gas velocity decreases, so that the upward-flowing particles leave the gas stream 23 laterally and drop back into the processing space 8. The processing space 8 is delimited in the lower region by sloping side walls 29. Due to this lateral slope, under the action of gravity the particles are carried via the return zone 24 toward the gas inlet 13 and the slit 1, where they are then entrained again by the process gas into the processing space 8. By this mechanism, a very uniform solids circulation 15 forms, consisting of an upward flow and return to the process gas inlet. Therefore even with very small amounts of particles in the processing space 8 there is a high particle density in the core zone above the deflector 3. One or more spray nozzles 7, which spray upward, parallel to the process gas stream, and serve for introducing the dispersion, are arranged in this region. Owing to the high particle loading in the core zone, very advantageous conditions for heat and mass transfer develop in the nozzling zone 22. Furthermore, the dispersion is mainly deposited on the particles and these are therefore wetted uniformly on the particle surfaces. The uniform wetting with simultaneous high solids circulation between the nozzling region and the return zone 24 has the effect that a very uniform liquid film is formed. As a result of the solidification process, the dispersion solidifies and the solid remains on the particle surface. In consequence, the granules grow very uniformly and homogeneously, which leads to a very narrow grain size distribution and a homogeneous particle structure. The process gas can entrain a proportion of the particles and fine material and dust as solid-laden waste air 20 from the processing space 8. The filter system optionally integrated in air outlet 19 or the dust-collecting equipment mounted after the apparatus can be used for separating these particles. In the case of an integrated dust collector 25, for example, compressed air pulses 18 can be used for returning the retained particles as separated solid 21 to the processing space 8.

Compared with fluidized-bed apparatus with integrated filtration equipment, dust return is facilitated because the upward-directed process gas flow is essentially spatially limited and therefore the particles that are to be returned can descend more reliably outside of the gas stream. This mechanism is additionally promoted by the suction effect in the vicinity of the gas inlet slit 1. Alternatively, particles separated from the waste air can be returned to the processing space 8. For this, various kinds of feeders 26 can be arranged in the lower region of the sloping side walls 29.

Owing to the high velocity of the process gas stream in the vicinity of the gas inlet slit 1, the fine particles are sucked in and delivered to the nozzling zone 22, where they are wetted with dispersion and take part in the growth process.

Optionally fitted baffles 16 support the gas stream, intensify the suction effect and improve the delivery of the solids into the nozzling zone 22. Any agglomeration effects occurring are minimized, because very high flow velocities, and therefore higher separating forces than in fluidized beds, occur in the nozzling region. As a result, particles are separated and grow into granules with a spherical shape. The flow profile of the process gas in the processing space 8 means, moreover, that fine particles returned by the optionally integrated filtration plant to the processing space do not drop back into the nozzling zone 22. This prevents the sticking together of fine particles and the resultant agglomerate forming processes. For a continuous process, the apparatus can optionally be equipped with various feed systems 13 for solids. This means for example that particles can be supplied to the process that can be obtained by comminution of for example (excessively large) granules and/or consist of granules that are too small. These particles then serve as granulation nuclei or as initial fill for shortening the start-up time. Moreover, additives that are to be embedded in the granules can be introduced in solid form into the process at this point. Furthermore, the apparatus can be provided with discharge devices 4, so that particles can be removed from the processing space 8. This can for example be effected with an overflow or with a volumetric discharge device (e.g. a rotary-vane feeder) or also with a gravity separator (e.g. a zigzag classifier supplied with classifying gas or a gravity classifier). Optionally, mechanical units 27 can be fitted in the processing space 8, though preferably in the region of the return zone 24 on the sloping walls, in order to produce, by comminution, sufficient fine material as nuclei for the granule formation process. Furthermore, the return zone 24 can optionally be used for positioning heating devices or other heat transfer devices 28. For example, the wall of the apparatus can be of double-walled construction, for use for heating or cooling the walls, for example using liquid or gaseous heat transfer agents. In this way, optimal surface temperatures can be provided, for example to prevent product deposits. In the processing space 8 or in the parts of the apparatus above that, the expansion zone 14 and the air outlet 19, optionally spray nozzles 6 can be arranged, preferably spraying downward but also partially upward. The liquid formulation can also be injected here, in order for example to produce granulation nuclei by spray drying/spray solidification in the apparatus. Alternatively, additives or other components in liquid form can be injected via some of the spray devices 6 and 7 and can therefore be embedded homogeneously in the granule structure. If the spray nozzles pass the temperature-adjusted ingoing air chamber 17, optionally the liquid-conveying parts can be provided with insulation or different cooling or heating systems 12, to prevent damage to the liquid formulation.

As a further advantage of the process according to the invention we should mention the very simple construction, which combines high operational reliability and insensitivity to disturbances with very easy cleaning. Therefore improved production conditions are created, in particular with respect to the pharmaceutical and hygiene requirements with product changes.

The pellets produced with the method according to the invention are preferably inert, nonhygroscopic and spherical and have a smooth surface. They preferably possess high mechanical strength, high density and display little abrasion. They have a narrow particle distribution, and the average particle diameter can be set as desired. In particular they are suitable as starter pellets.

The pellets according to the invention can for example be sprayed in a fluidized bed with a dispersion containing an active substance for the purpose of active substance layering, i.e. to build up a layer of active substance. For building up the layer of active substance, the dispersion of active substance can contain the active substance in dissolved, suspended or emulsified form. Preferably the active substance is applied in dissolved or suspended form on the pellet according to the invention. Water or alcohols such as methanol, ethanol or isopropanol or ketones such as acetone and mixtures of water and the organic solvents just described are suitable as solvents and suspending agents. Binders, separating agents, stabilizers or other excipients can be added to the dispersant described. Following application of the layer of active substance, a functional coating can be applied for stabilization, masking of taste or modification of the release properties. In the latter case, for example gastric-resistant films, layers that modify i.e. delay the release of the active substance or sustained-release coatings can be applied on the pellets.

Optionally an interlayer of water-soluble polymers or water-soluble substances and other excipients can be interposed between the active substance and functional coating. Finally, the coated pellet thus obtained can contain an outer separating layer or an external phase.

The coating operations described here are typically carried out in a fluidized bed.

The pellets that are coated with active substance as has been described above are in a preferred embodiment pellets that contain mannitol, and in particular, pellets consisting essentially of mannitol.

The active substances are preferably chemically labile or unstable and moisture-sensitive pharmaceutical active substances, for example duloxetine or a pharmaceutically compatible salt thereof, such as duloxetine hydrochloride, or active substances from the group of proton pump inhibitors, such as e.g. pantoprazole, esomeprazole, omeprazole, lansoprazole, rabeprazole and salts thereof.

According to one aspect of the invention, pellets are provided that have a mannitol core and a layer of active substance, in which the active substance is duloxetine or a pharmaceutically compatible salt thereof, preferably duloxetine hydrochloride.

Special embodiments of the invention are presented below:

1. Pellets containing a pharmaceutical substance, characterized in that the pellets have a breaking strength of more than 0.001 newton.
2. Pellets according to Point 1, characterized in that the breaking strength is between 0.05 newton and 10 newton.
3. Pellets according to Point 1 or 2, characterized in that the weight average particle diameter is 0.1 to 3 mm.
4. Pellets according to one of Points 1 to 3, characterized in that the length-width ratio of the pellets is less than about 1.4, preferably less than about 1.3, more preferably less than about 1.2, even more preferably less than about 1.1 and in particular less than about 1.05.
5. Pellets according to one of Points 1 to 4, characterized in that the pharmaceutical substance contains an excipient.
6. Pellets according to one of Points 1 to 5 containing a water-soluble and/or nonhygroscopic and sucrose-free excipient.
7. Pellets according to one of Points 1 to 6 containing a sucrose-free excipient.
8. Pellets according to one of Points 1 to 7 containing mannitol.
9. Pellets according to one of Points 1 to 8, characterized in that the pharmaceutical substance contains a pharmaceutical active substance.
10. Pellets according to one of Points 1 to 9 constructed from a mannitol core and a layer applied thereon, which contains a pharmaceutical active substance.
11. Method of production of pellets containing a pharmaceutical substance, preferably with a breaking strength of more than 0.001 N, comprising the stages:
(a) preparation of a solution or dispersion of a pharmaceutical substance;
(b) production of droplets by spraying the solution or dispersion into a processing space;
(c) repeated directing of solid particles past injected droplets in the processing space by means of a process gas stream conveyed in a preferentially defined manner;
(d) removing particles from the processing space in relation to particle size.
   12. Method according to Point 11, characterized in that in step (b) a solution or dispersion is injected at a temperature of 60 to 100° C.
   13. Method according to Point 11 or 12, characterized in that the solution or dispersion contains an organic solvent, a mixture of an organic solvent and water or water as solvent, water being preferred.
   14. Method according to one of Points 11 to 13, characterized in that a solution is used that is saturated or supersaturated at the injection temperature selected in step (b).
   15. Method according to one of Points 11 to 14, characterized in that a solution or dispersion of a water-soluble and/or nonhygroscopic excipient is used.
   16. Method according to one of Points 11 to 15, characterized in that a solution or dispersion of a sucrose-free excipient is used.
   17. Method according to one of Points 11 to 16, characterized in that a solution or dispersion containing mannitol is used.
   18. Method according to one of Points 11 to 17, characterized in that, in the processing space, a spouted bed is formed from fluidized solid particles.
   19. Method according to Point 18, characterized in that solid particles are led past injected droplets, with droplets of the dispersion being injected into the spouted bed.
   20. Method according to Point 18 or 19, characterized in that the droplets of the dispersion are injected into the spouted bed in the region of the ejection zone.
   21. Method according to one of Points 11 to 20, characterized in that the droplets are injected in parallel with the process gas stream.
   22. Method according to one of Points 11 to 21, characterized in that the droplets are injected into the ejection zone in the region adjacent to the return zone.
   23. Method according to one of Points 11 to 22, characterized in that the temperature of the process gas stream that is conveyed in a defined manner is between 10° C. and 150° C., especially between 30 to 120° C., and preferably between 60° C. and 110° C.
   24. Method according to one of Points 11 to 23, characterized in that solid particles are introduced into the processing space from outside.
   25. Pellets, produced by a method according to one of Points 11 to 24.
   26. Use of pellets according to the definition in one of Points 1 to 10 or 25 for the production of a pharmaceutical dosage form, in particular for the production of a tablet, a capsule or a sachet.

### EXAMPLES

The invention will be illustrated with concrete examples of application, without being in any way restricted to these.

### Example 1

### Production of Mannitol Pellets

A mannitol solution at a temperature of 90° C.-100° C. was injected into an apparatus that is characterized by the construction described previously. The processing space is characterized by a rectangular cross-section and has, above the sloping side walls, a cross-sectional area of 0.25×0.2=0.05 m² and a height of about 1 m. The process air stream, preheated to about 75° C., was supplied at about 100 m³/h via two gas supply slits running longitudinally through the apparatus. The dispersion was injected at an air pressure of 2.3 bar via a two-component nozzle supplied with compressed air, spraying vertically upward into the process air jet. There was about 500 g of mannitol powder in the processing space.

| Parameter | Unit | Value |
|---|---|---|
| Inlet air amount | m³/h | 100 |
| Inlet air | ° C. | 95 |
| temperature | | |
| Outlet air | ° C. | 57 |
| temperature | | |
| Product temperature | ° C. | 67 |
| Spray nozzle | mm | 1.2 |
| diameter | | |
| Spray pressure | bar | 3.0 |
| Classifier air | bar | 0.7 |
| pressure | | |
| Spraying rate | g/min | 40 |

At this pressure setting, the classifier provided mainly material with a grain fraction between 160 μm and 315 μm. The pellets isolated in this way had a bulk density of about 0.7 g/ml. The mannitol pellets were very round, of uniform grain distribution, very hard and had a smooth, shiny surface.

### Example 2

### Production of Mannitol Pellets

A mannitol solution at a temperature of 70-80° C. was injected into an apparatus that is characterized by the construction described previously. The processing space is characterized by a rectangular cross-section and has, above the sloping side walls, a cross-sectional area of 0.25×0.2=0.05 m² and a height of about 1 m. The process air stream, preheated to about 75° C., was supplied at about 100 m³/h via two gas supply slits running longitudinally through the apparatus. The dispersion was injected at an air pressure of 2.3 bar via a two-component nozzle supplied with compressed air, spraying vertically upward into the process air jet. There was about 500 g of mannitol powder in the processing space.

| Parameter | Unit | Value |
|---|---|---|
| Inlet air amount | m³/h | 100 |
| Inlet air | ° C. | 75 |
| temperature | | |
| Outlet air | ° C. | 41 |
| temperature | | |
| Product temperature | ° C. | 50 |
| Spray nozzle | mm | 1.2 |
| diameter | | |
| Spray pressure | bar | 2.3 |
| Classifier air | bar | 0.9 |
| pressure | | |
| Spraying rate | g/min | 50 |

At this pressure setting, the classifier provided mainly material with a grain fraction between 315 μm and 500 μm. The pellets isolated in this way had a bulk density of about 0.7 g/ml. The mannitol pellets were smooth, round and hard.

### Example 3

### Production of Duloxetine-Containing Pellets Description of Active Substance Layering

Duloxetine hydrochloride is dissolved completely (11.25% w/w solid) in a mixture of ethanol and water 50:50 w/w. Methocel E6 LV is added to the resultant solution (15.00% w/w total solids). This solution is sprayed onto mannitol pellets with a grain size of 315-500 μm (75% weight increase). For this, the mannitol pellets are fluidized in a fluidized-bed apparatus of appropriate size (e.g. Glatt GPCG 1) with Wurster insert and are then sprayed with the active substance solution. The solution is injected at an air pressure of 2.0 bar. The processing space contains about 400 g of mannitol pellets. The production of suitable mannitol pellets is described in the above examples. The process conditions are given below:

| Parameter | Unit | Value |
|---|---|---|
| Inlet air amount | m³/h | 150 |
| Inlet air | ° C. | 47 |
| temperature | | |
| Outlet air | ° C. | 40 |
| temperature | | |
| Product temperature | ° C. | 40 |
| Spray nozzle | mm | 1.2 |
| diameter | | |
| Spray pressure | bar | 2.0 |
| Spraying rate | g/min | 10 |

### Example 4

### Stability Investigations

We were able to demonstrate that pellets containing duloxetine hydrochloride with mannitol as starter core have greater chemical stability than pellets containing duloxetine hydrochloride that have a starter core of microcrystalline cellulose of comparable grain size. Pellets containing duloxetine hydrochloride with microcrystalline cellulose as starter material were produced under the same conditions as described above. The chemical stability of the two samples was investigated by HPLC immediately after production. The following table compares the chemical stability of the different batches of duloxetine hydrochloride. The method of analysis is briefly explained below.

### HPLC Method for Determination of Related Substances

Chromatography Conditions:
HPLC equipment: Rapid Resolution System, Series 1200 from Agilent Technologies
Detector: DAD
Column: Zorbax Eclipse XDB-C18, 50 mm×4.6 mm, 1.8 μm grain size
Pre-column: C18 4×3 mm
Wavelength: 220 nm
Column temperature: 30° C.
Development time: 3 min.
Injection volume: 5 μl

### Preparation of the Buffer Solution for Eluent:

Dissolve 0.5 ml HPLC-pure trifluoroacetic acid in a 1000 ml graduated flask in HPLC-pure water and make up to the mark with HPLC-pure water. Filter the buffer with a 0.45 μm filter. The HPLC method runs “isocratically” with a mixture ratio of buffer to acetonitrile of 65:35 (v/v).

### Solvent for Standard and Samples:

Standard and samples are prepared at mixture ratio water/acetonitrile 50:50 (v/v).

An accurately weighed amount of standard sample is weighed in a 250 ml brown glass graduated flask. Add 200 ml of the water/acetonitrile solvent and put the samples in an ultrasonic bath for approx. 15 min. After cooling to room temperature, the sample solutions are made up to the mark with the solvent, filtered with a 0.22 μm PVDF filter into brown vials, the first 2 ml being discarded, and are then injected.

| | Related substances | | | | | | | |
|---|---|---|---|---|---|---|---|---|
| | | | | R- | N-methyl | | | |
| | | Unknown | Unknown | duloxetine | duloxetine | Unknown | | |
| | Phenol | component 1 | component 2 | hydrochloride | oxalate | component 3 | β-Naphthol | Total |
| Designation | (%) | (%) | (%) | (%) | (%) | (%) | (%) | (%) |
| Relative | 0.47 | 0.63 | 0.94 | 1.08 | 1.21 | 1.43 | 1.63 | — |
| retention time | | | | | | | | |
| S-Duloxetine HCl | 0.055 | 0.052 | — | — | 0.003 | — | — | 0.110 |
| coated pellets, | | | | | | | | |
| starter core of | | | | | | | | |
| microcrystalline | | | | | | | | |
| cellulose (batch | | | | | | | | |
| SDF 2151) | | | | | | | | |
| S-Duloxetine HCl | — | — | — | — | — | — | — | 0.000 |
| coated pellet | | | | | | | | |
| with starter core | | | | | | | | |
| of mannitol | | | | | | | | |
| pellets (batch | | | | | | | | |
| SDF 2170) | | | | | | | | |

### REFERENCE NUMBERS

1 slit(s)
2 gas jet(s)
3 deflector
4 discharge device
5 side wall
6 spray nozzle(s) spraying in any directions
7 spray nozzle(s) spraying upward
8 processing space
9 cross-section of a processing stage
10 process gas
11 waste gas
12 insulation with cooling or heating system
13 inlet system
14 expansion zone
15 solids circulation
16 baffle(s)
17 ingoing air chamber
18 compressed air pulses
19 air outlet
20 solids-laden waste air
21 separated and returned solids
22 nozzling zone
23 exit of particles from the gas jet
24 return zone
25 dust collector
26 supply lines

1. Pellets containing a pharmaceutical substance, characterized in that the pellets have a breaking strength of more than 0.001 newton, preferably between 0.05 newton and 10 newton.

2. Pellets according to claim 1, characterized in that the weight average particle diameter is 0.1 to 3 mm.

3. Pellets according to claim 1, characterized in that the length-width ratio of the pellets is less than about 1.4, preferably less than about 1.3, more preferably less than about 1.2, even more preferably less than about 1.1 and in particular less than about 1.05.

4. Pellets according to claim 1, containing a water-soluble and/or nonhygroscopic and sucrose-free excipient, characterized in that it is preferably mannitol.

5. Pellets according to claim 1, built up from a core not containing active substance, in particular a mannitol core, and a layer that contains a pharmaceutical active substance.

6. Method of production of pellets containing a pharmaceutical substance, preferably with a breaking strength of more than 0.001 N, comprising the stages:
(a) preparation of a solution or dispersion of a pharmaceutical substance;
(b) production of droplets by spraying the solution or dispersion into a processing space;
(c) repeated directing of solid particles past injected droplets in the processing space by means of a process gas stream conveyed in a preferentially defined manner;
(d) removing particles from the processing space in relation to particle size.

7. Method according to claim 6, characterized in that a spouted bed is formed from fluidized solid particles in the processing space.

8. Method according to claim 7, characterized in that solid particles are directed past injected droplets by injecting droplets of the dispersion into the spouted bed, preferably in the region of the ejection zone of the spouted bed.

9. Method according to claim 6, characterized in that the droplets are injected parallel to the process gas stream.

10. Method according to claim 6, characterized in that the droplets are injected into the ejection zone in the region adjacent to the return zone.

11. Pellets produced by a method according to claim 6.

12. Use of pellets as defined in claim 1 for the production of a pharmaceutical dosage form, in particular for the production of a tablet, a capsule or a sachet.
